(19) United States
(12) Patent Application Publication
(10) Pub. No.: US 2008/0311631 A1
(43) Pub. Date: Dec. 18, 2008

(54) Biosynthetic Production of 4-Amino 4-Deoxychorismate (Adc) and [3R,4R]-4-Amino-3-Hydroxycyclohexa-1,5-Diene-1-Carboxylic Acid (3,4-Cha)

(76) Inventors: Marcel Gerhardus Wubbolts, Sittard (NL); Roelof Ary Lans Bovenberg, Rotterdam (NL); Georg Sprenger, Backnang-Maubach (DE); Johannes Josef Bongaerts, Leverkusen (DE); Stefan Kozak, Stuttgard (DE); Michael Muller, Julich (DE); Volker Lorbach, Duren (DE)

Correspondence Address: NIXON & VANDERHYE, PC, 901 NORTH GLEBE ROAD, 11TH FLOOR, ARLINGTON, VA, 22203, US

(21) Appl. No.: 11/628,459

(22) PCT Filed: Jun. 2, 2005
(86) PCT No.: PCT/EP05/05988

§ 371 Date: Mar. 15, 2007

(30) Foreign Application Priority Data
Jun. 4, 2004 (EP) 04076644.6

(51) Int. Cl. C12P 13/22; C12P 13/00; C12N 15/63; C12N 1/20; C07C 61/22

(52) U.S. Cl. 435/108; 435/128; 435/320.1; 435/252.5; 435/252.1; 435/252.8; 562/400

(57) ABSTRACT

The invention relates to a process for the biosynthetic production of 4-amino-4-deoxychorismate (ADC) performed fermentatively in vivo with a 4-amino-4-deoxychorismate synthase, preferably a PabAB bipartite protein (which may be a fusion protein), at an increased level of activity, thereby obtaining a broth comprising ADC and 4-amino-4-deoxyprephenate (ADP), that are recovered. The invention also relates to a further process of converting the ADP into p-aminophenylalanine. The invention, moreover relates to biosynthetic production of [3R,4R]-4-amino-3-hydroxycyclohexa-1,5-diene-1-carboxylic acid (3,4-CHA), by concerted action of such 4-amino-4-deoxychorismate synthase and of an enzyme capable of converting isochorismate into [5S,6S]-5,6 dihydroxycyclohexa-1,3-diene-1-carboxylic acid (2,3-CHD), preferably a phenazine biosynthesis protein PhzD, including recovery of 3,4-CHA. The invention also relates to expression vectors and host cells for use in any of such processes. The invention further relates to the use of 3,4-CHA as a catalytically active product, in particular as a chiral catalyst. And the invention finally relates to synthesis of oseltamivir phosphate from 3,4-CHA.

The present invention relates, in a first embodiment thereof, to a process for the biosynthetic production of 4-amino-4-deoxychorismate (ADC) catalyzed at least by an enzyme belonging to the class of aminodeoxychorismate synthases. In said process a mixture of 4-amino-4-deoxychorismate (ADC) and 4-amino-4-deoxyprephenate (ADP) is formed in a fermentation broth. In an integrated subsequent step of said embodiment, the invention also relates to the synthesis of p-aminophenylalanine.

The term biosynthetic production as used herein is used in its broadest possible meaning, unless it is clear from the context that a narrower meaning is intended. It includes processes that are carried out fermentatively in vivo, as well as processes that are carried out in vitro. Generally, in vivo processes are processes carried out when using living cells (the term “living cells” thereby also including so-called resting cells); in vitro processes, on the other hand, usually are carried out using cell lysates or (partly) purified enzymes. The biochemical production as meant herein, however, also may be carried out using permeabilized cells; the differentiation between in vivo and in vitro, however, does not make much sense for processes being carried out with permeabilized cells. It will be evident, that the process of the invention also can be carried out using immobilized host cells, immobilized enzymes, etc. In case claims are restricted to “in vivo” processes, this will be indicated specifically. Although, strictly speaking from a technical point of view, the term “fermentatively” often would mean that a process is carried out without oxygen or under limited oxygen supply, the term is used in the context of the present patent application in the broadest possible sense and includes aerobic processes as well. It intends to indicate that all general techniques available for the fermentation technologist can be used in the methods according to the present invention.

The present invention also, in a second embodiment, relates to a process for the biosynthetic production of [3R,4R]-4-amino-3-hydroxycyclohexa-1,5-diene-1-carboxylic acid (3,4-CHA). This compound [3R,4R]-4-amino-3-hydroxycyclohexa-1,5-diene-1-carboxylic acid (3,4-CHA) can also be referred to as trans-4-amino-3-hydroxycyclohexa-1,5-diene-1-carboxylic acid.

Further, the present invention also relates to host cells, expression vectors, plasmids and so on as can be used in the processes of any of the embodiments of the present invention.

In a still further embodiment, the present invention relates to the use of [3R,4R]-4-amino-3-hydroxycyclohexa-1,5-diene-1-carboxylic acid (3,4-CHA) as a catalytically active product, in particular as a chiral catalyst.

Finally, the present invention also relates to the further conversion of [3R,4R]-4-amino-3-hydroxycyclohexa-1,5-diene-1-carboxylic acid (3,4-CHA), as can be produced according to the present invention. In particular, it relates to the further conversion in a novel synthesis route for the production of a product called oseltamivir phosphate (IUPAC name: ethyl (3R,4R,5S)-4-N-acetyl-amino-3-(1-ethylpropoxy)-5-N-amino-1-cyclohexene-1-carboxylate phosphate [1:1]), which is the active ingredient for an anti-influenza pharmaceutical product known under the trade name Tamiflu® of Hoffmann-La Roche, Switzerland.

Biosynthetic production of 4-amino-4-deoxychorismate (ADC) is known from K. S. Anderson et al., JACS 113 (1991), pages 3198-3200. Parsons et al., in Biochem. 42, p. 5684-5693 (2003), describe at page 5690 that ADC is only barely hydrolyzed under the influence of the phenazine biosynthesis PhzD protein for which it apparently is a poor substrate.

Biosynthesis of [3R,4R]-4-amino-3-hydroxycyclohexa-1,5-diene-1-carboxylic acid (3,4-CHA) so far has not been described at all. For the synthesis of oseltamivir phosphate so far only quite laborious methods are available comprising more than 10 reaction steps. Reference, for instance can be made to a 12-step method starting from quinic acid, as described by Rohloff et al., J. Org. Chem., 63, p. 4545-4550 (1999); or to an 11-step method starting from shikimic acid as described by M. Federspiel et al., Organic Process Research & Development, 3, p. 266-274 (1999).

Although the biosynthesis route to 4-amino-4-deoxyprephenate (ADP) is known, for instance from Teng et al., J. Am. Chem. Soc., Vol. 107, p. 5008-5009 (1985), biosynthetic production and recovery of ADP has never been described, presumably because the product ADP, like it is known for ADC, is believed to be unstable. This reference of Teng et al., similar to the disclosure of Blanc et al., Mol. Mic., Vol. 23, p. 191-202 (1997), also shows a possible biosynthesis route to aminodeoxychorismate (ADC), respectively to p-aminophenylalanine, but no fermentative route and recovery of the products ADP and ADC, respectively of p-aminophenylalanine, is suggested.

Disadvantages of the known methods are:

a) for the biosynthetic production of aminodeoxychorismate (ADC) so far only synthesis at mg-scale, and at very low concentrations of a few mg/l, has been described; thus, recovery of ADC from such reaction mixtures is very difficult;
b) for the biosynthetic production of [3R,4R]-4-amino-3-hydroxycyclohexa-1,5-diene-1-carboxylic acid (3,4-CHA) no method is yet available at all;
c) for the state-of-the-art synthesis of oseltamivir phosphate so far a more than 10 step reaction sequence is needed.

Accordingly there is need for an improved process for the biosynthesis of aminodeoxychorismate (ADC), for a completely novel biosynthetic process for the synthesis of [3R,4R]-4-amino-3-hydroxycyclohexa-1,5-diene-1-carboxylic acid (3,4-CHA), and for an improved process for the synthesis of oseltamivir phosphate.

The above problems all have been solved by the present invention, having the basic features as are claimed in claim 1 hereof.

The present inventors, surprisingly, now have found that the biosynthetic production of 4-amino-4-deoxychorismate (ADC), catalyzed at least by an enzyme belonging to the class of aminodeoxychorismate synthases, is performed fermentatively in vivo in a host microorganism with a 4-amino-4-deoxychorismate synthase at an increased level of activity, while obtaining a fermentation broth comprising 4-amino-4-deoxychorismate (ADC) and 4-amino-4-deoxyprephenate (ADP), and that these compounds, either together or individually, are recovered from the fermentation broth.

Because ADC synthesis in nature is the first step in folate synthesis from chorismate, aminodeoxychorismate synthase enzymes are abundantly available in nature. They are assumed to be present in all folate prototrophic organisms, e.g. in bacteria, yeasts, plants and lower eukaryotes. Aminodeoxychorismate synthase enzymes are also known to be involved in p-aminobenzoate synthesis.

As used herein, the term “at an increased level of activity” means that the level of activity (of any particular enzyme as indicated) is higher than the level of (native) activity of said enzyme in its native surroundings (i.e. natural source cells) under standard conditions. Increasing the activity level can be achieved by all kinds of methods known to the skilled man, e.g. by overexpression of the gene coding for such enzyme, multi-copying of such genes, or providing the genes with improved translational and/or transcriptional efficiency, for instance by means of a stronger promoter, an inducible promoter, etc.

ADC synthases, and in any case those from Escherichia coli, are known to exist in the form of single units, but sometimes occur as bipartite enzymes. The single units, for instance, may consist of a so-called PabA protein (a protein known to generate ammonia from glutamine) or of a so-called PabB protein (a protein known to insert ammonia directly into chorismate).

G. Bassett et al. describe in PNAS, 101, pages 1496-1501 (2004), that bipartite proteins containing fused PabA and PabB domains are involved in the synthesis of ADC in plants. It is to be noted, that—as is also done in the article of Bassett et al., these ADC synthases in literature occasionally (and, as the present inventors believe, wrongly) are also referred to as PABA synthases (p-aminobenzoate synthases). The present inventors have shown in their studies that enzymes, which incorrectly have been named PABA synthases, indeed all lead to the formation of ADC (in admixture with ADP), and that formation of p-aminobenzoate (if observed at all; it is at most as a minor by-product) hardly occurs in the process embodiments of the present invention. As Bassett et al. have confirmed, the conversion into p-aminobenzoate requires the additional presence of the protein PabC. Accordingly it is most surprising in the methods of the present invention, that, although the pabC may be present, no p-aminobenzoate is formed.

In a preferred embodiment of this first embodiment of the invention the biosynthetic production of 4-amino-4-deoxychorismate (ADC) is performed whereby the 4-amino-4-deoxychorismate synthase is a PabAB bipartite protein.

As meant herein, the term 4-amino-4-deoxychorismate synthase PabAB bipartite protein represents any active protein (i.e. any protein having the functionality of an ADC synthase) that combines the functions of PabA (a protein known to generate ammonia from glutamine) and PabB (a protein known to insert ammonia directly into chorismate). In principle, the term “bipartite protein” as used herein interchangeably can be used with any of the terms “fusion protein” or “covalently linked protein complex”.

The process according to the first embodiment of the invention is carried out in vivo in a host microorganism. In the processes of the prior art only in vitro synthesis is described.

In particular, and preferably, the bipartite protein originates from a species from the group of Actinomycetes, or from plants containing such bipartite enzyme, or is constructed by fusion of genes respectively encoding for PabA and PabB. Methods for fusion of genes are well known to the skilled man, and may, for instance, consist of PCR methods, cloning, etc.

Accordingly, the bipartite protein may be a naturally occurring (bipartite) protein from plants, or an artificially constructed one. The inventors have observed that there is rather high homology between PabA proteins and certain glutamine aminotransferases that are also referred to as TrpD proteins, and that there is also rather high homology between PabB proteins and certain anthranilate synthases that are also referred to as TrpE proteins. Accordingly—instead of, or together with—the PabA resp. PabB parts of the bipartite protein, also TrpD (instead of PabA) and TrpE (instead of PabB) can be applied in the process of this invention.

This means that any of the following combinations PabA/PabB; TrpD/PabB; PabA/TrpE; and TrpD/TrpE

all lead to favorable results according to (this first embodiment on the present invention. Therefore, all such combinations of enzyme activities are encompassed in the term 4-amino-4-deoxychorismate synthase PabAB bipartite protein.

Because TrpD and TrpE in nature are involved in the first steps of tryptophan synthesis these enzymes are abundantly available in nature. They are assumed to be present in all L-tryptophane prototrophic organisms, e.g. in bacteria, yeasts, plants and lower eukaryotes.

Accordingly, genes coding for each of the parts of the bipartite proteins PabA/PabB; TrpD/PabB; PabA/TrpE; and TrpD/TrpE are well-known and available to the skilled man.

Preferably, the PabAB bipartite protein originates, as such or as a fusion protein, from a species from the group of genera consisting of Escherichia, Corynebacterium, Saccharomyces or Streptomyces. In the process according to the invention for the biosynthesis of ADC, the PabAB bipartite protein more preferably originates, as such or as a fusion protein, from one of the species from the group of species consisting of Escherichia coli, Corynebacterium glutamicum, Corynebacterium diphtheriae gravis NCTC13129, Corynebacterium efficiens, Saccharomyces cerevisiae, Streptomyces griseus, Streptomyces venezuelae, Streptomyces sp. FR-008 (these are strains involved in the synthesis of polyketide FR-008), Streptomyces pristinaespiralis, Streptomyces thioluteus, and Streptomyces avermitilis. An example of a Streptomyces sp. FR-008 strain is the strain deposited, on 20 Oct. 2003, at the Korean Collection for Type Cultures under KCTC 10529BP. It is most preferred, that the PabAB bipartite protein originates from Corynebacterium glutamicum ATCC 13032.

As mentioned above, the proteins are to be present “at an increased level of activity”. This means that the level of activity (of any particular enzyme as indicated) is higher than the level of (native) activity of said enzyme in its native surroundings (i.e. natural source cells) under standard conditions.

Best results are achieved when expression of the PabAB bipartite protein is manipulated in such way that timing of the start of actual production is started after the host microorganism in which the protein is expressed has reached an O.D. (optical density) at 620 nm in the range of from about 0.5 to 100, preferably of at most 50. This expression can be achieved with or without induction. In case the expression is achieved with induction, it is preferably done with help of a strong promoter, for instance a ptac promoter, and is induced by isopropyl-β-D-thiogalactopyranoside (IPTG).

In this first embodiment of the present invention, the 4-amino-4-deoxy-chorismate (ADC) and 4-amino-4-deoxyprephenate (ADP) formed are preferably recovered from the said obtained fermentation broth, together or individually, by a separation process selected from the group consisting of reactive extraction and chromatography, optionally followed by crystallization.

In a subsequent and integrated version of this first embodiment of the invention, the synthesis of p-aminophenylalanine is achieved. This is done by biosynthetic production of p-aminophenylalanine integrated in a biosynthetic process for the production of 4-amino-4-deoxychorismate (ADC) catalyzed at least by an enzyme belonging to the class of aminodeoxychorismate synthases, wherein the biosynthetic production is performed fermentatively in vivo in a host microorganism with a 4-amino-4-deoxychorismate synthase at an increased level of activity, while obtaining a fermentation broth comprising 4-amino-4-deoxychorismate (ADC) and 4-amino-4-deoxyprephenate (ADP), and wherein the 4-amino-4-deoxyprephenate (ADP) in the mixture of 4-amino-4-deoxychorismate (ADC) and 4-amino-4-deoxyprephenate (ADP) is converted into p-aminophenylalanine by means of concerted action, and at an increased level of activity, of a 4-amino-4-deoxyprephenate dehydrogenase and an aminotransferase, and wherein that p-aminophenylalanine is recovered from the fermentation broth.

The aminotransferase may be an L- or D-specific aminotransferase from any suitable source. Of course, the p-aminophenylalanine then may be converted in subsequent steps into derivatives thereof.

The 4-amino-4-deoxyprephenate dehydrogenase is preferably a PapC protein. The gene papC is, for instance, known from the pristinamycin biosynthesis in Streptomyces pristinaespiralis (see Blanc et al., Molec. Microbiol., 23, p. 191-202 (1997). The papC gene has a close resemblance with the cmlC gene (also encoding for 4-amino-4-deoxyprephenate dehydrogenase) as is involved in chloramphenicol synthesis in Streptomyces venezuelae (see He et al., Microbiol., 147, p. 2817-2829 (2001). For the purposes of the present invention, proteins encoded by cmlC are also considered to be PapC proteins.

The conversion of ADP into p-aminophenylalanine is described in a paper of R. A. Mehl et al., JACS 125, p. 935-939 (2003), wherein (as shown in FIG. 1 at page 936) subsequent action of PapA, PapB and PapC followed by interaction with an E. coli aminotransferase leads to the formation of p-aminophenylalanine. It is to be noticed, that these authors indeed cloned the cml genes from S. venezuelae for the biosynthesis of p-aminophenylalanine, but they labeled these genes with pap as would have been done according to the labeling of the S. Pristinaespiralis genes. According to the above described embodiment of the present invention the number of enzymes involved, and thus the number of steps, in the conversion into p-aminophenylalanine is one lower than in the route described by Mehl et al.

The present invention, in a second embodiment thereof, relates to a process for the biosynthetic production of [3R,4R]-4-amino-3-hydroxycyclohexa-1,5-diene-1-carboxylic acid (3,4-CHA) catalyzed at least by an enzyme belonging to the class of aminodeoxychorismate synthases, wherein the biosynthetic production is performed by concerted action, and at an increased level of activity, of a 4-amino-4-deoxy-chorismate synthase and of an enzyme capable of converting isochorismate into [5S,6S]-5,6-dihydroxycyclohexa-1,3-diene-1-carboxylic acid (2,3-CHD), and wherein the [3R,4R]-4-amino-3-hydroxycyclohexa-1,5-diene-1-carboxylic acid (3,4-CHA) is recovered from the fermentation broth so obtained. 2,3-CHD also can be referred to as [2S,3S]-2,3-dihydroxy-2,3-dihydrobenzoic acid.

In this embodiment of the invention, the biosynthetic production may be carried out either fermentatively in vivo in a host microorganism, or may be carried out enzymatically in vitro, for instance by using enzyme preparations comprising the aforementioned enzyme activities. Such enzyme preparations, for instance, may be present in the form of enzymes on carrier, or in resting cells, or as cell lysates, or as (partly) purified enzymes, or in any other form known to the skilled man.

As meant herein, the phrase “by concerted action, and at an increased level of activity” indicates that both enzymes mentioned act together, and each of them is being used at a level of activity (of such enzyme) that is higher than the level of (native) activity of said enzyme in its native surroundings (i.e. natural source cells) under standard conditions. As indicated before, increasing the activity level of an enzyme can be achieved by all kinds of methods known to the skilled man, e.g. by overexpression of the gene coding for such enzyme, multi-copying of such genes, or providing the genes with improved translational and/or transcriptional efficiency, for instance by means of a stronger promoter, an inducible promoter, etc.

It is noted that, in principle, and from a chemical point of view, ADC might be considered to be a mono-substituted (i.e. protected) form of [3R,4R]-4-amino-3-hydroxycyclohexa-1,5-diene-1-carboxylic acid (3,4-CHA). In fact, conversely, ADC also might be called a derivative of 3,4-CHA. ADC and 3,4-CHA both are important intermediates for the synthesis of interesting further compounds.

Preferably, the biosynthetic production of [3R,4R]-4-amino-3-hydroxy-cyclohexa-1,5-diene-1-carboxylic acid (3,4-CHA) is performed fermentatively in vivo in a host microorganism.

Suitable enzymes capable of converting isochorismate into [5S,6S]-5,6-dihydroxycyclohexa-1,3-diene-1-carboxylic acid (2,3-CHD) can be obtained from the enzyme class of isochorismatases [EC 3.3.2.1]. Of course, also mutants and muteins of this class of proteins that have been engineered so as to be optimized for the desired conversion of ADC into 3,4-CHA, even if they are no longer capable of catalyzing the conversion of isochorismate into 2,3-CHD, are—within the context of the present application—considered to fall within the said class of enzymes.

In this second embodiment of the present invention, the 4-amino-4-deoxy-chorismate synthase most preferably is a PabAB bipartite protein. For the PabAB bipartite protein all remarks are applicable as have been made in the foregoing part of this patent application with respect to the first embodiment of the invention.

Preferably, the enzyme capable of converting isochorismate into [5S,6S]-5,6-dihydroxycyclohexa-1,3-diene-1-carboxylic acid (2,3-CHD) is an isochorismatase enzyme.

In particular, in this embodiment, the PabAB bipartite protein is a protein as described above for the first embodiment of the invention, and the enzyme capable of converting isochorismate into [5S,6S]-5,6-dihydroxycyclohexa-1,3-diene-1-carboxylic acid (2,3-CHD) is also capable of converting 4-amino-4-deoxyisochorismate into [5S,6S]-6-amino-5-hydroxycyclohexa-1,3-diene-1-carboxylic acid (2,3-CHA).

[5S,6S]-6-amino-5-hydroxycyclohexa-1,3-diene-1-carboxylic acid (2,3-CHA) can also be referred to as [2S,3S]-2-amino-3-hydroxy-2,3-dihydrobenzoic acid, or as trans-2,3-dihydro-3-hydroxyanthranilic acid.

It is most preferred, that the enzyme capable of converting isochorismate into [5S,6S]-5,6-dihydroxycyclohexa-1,3-diene-1-carboxylic acid (2,3-CHD) is a phenazine biosynthesis PhzD protein. [5S,6S]-5,6-dihydroxycyclo-hexa-1,3-diene-1-carboxylic acid (2,3-CHD) can also be referred to as [2S,3S]-2,3-dihydroxy-2,3-dihydrobenzoic acid.

Genes coding for PhzD are, for instance, known from an article of McDonald et al., JACS, 123, p. 9459-9460 (2001). The phenazine genes have been identified and sequences by D. Mavrodi et al., as described in J. Bacteriol. 180, p. 2541-2548 (1998).

It is, moreover, mentioned in an article of J. B Laursen et al. in Chem. Rev. 104, p. 1663-1685 (2004), entitled “Phenazine natural products: biosynthesis, synthetic analogues and biological activity”, that phenazine natural products are isolated primarily from Pseudomonas and Streptomyces, and a few other genera from soil or marine habitats. Examples of such other species are: Methanosarcina mazei Gö1, Pelagiobacter variabilis, Vibrio strains, Erwinia herbicola (this is a strain also being named as Pantoea agglomerans), Burkholderia phenazinium, Waksmania aerata, and Sorangium species. Accordingly, proteins homologous to PhzD preferably are obtained from any of such strains.

Thus, preferably, the phenazine biosynthesis PhzD protein originates from a species from the group of genera consisting of Pseudomonas, Pantoea, Streptomyces, and Erwinia. In particular, the phenazine biosynthesis PhzD protein originates from a species selected from the group consisting of Pseudomonas aeruginosa, Pseudomonas aureofaciens, Pseudomonas fluorescens, Pseudomonas chlororaphis, and Pantoea agglomerans species. Most preferably, the phenazine biosynthesis PhzD protein originates from Pseudomonas aeruginosa ATCC 17933.

In this second embodiment of the present invention, the PabAB bipartite protein originates, as such or as a fusion protein, from a species from the group of genera consisting of Escherichia, Corynebacterium, Saccharomyces or Streptomyces, most preferably selected from the group of species consisting of Escherichia coli, Corynebacterium glutamicum, Corynebacterium diphtheriae gravis NCTC13129, Corynebacterium efficiens, Saccharomyces cerevisiae, Streptomyces griseus, Streptomyces venezuelae, Streptomyces sp. FR-008, Streptomyces pristinaespiralis, Streptomyces thioluteus, and Streptomyces avermitilis.

Most preferably, the bipartite protein originates from Corynebacterium glutamicum ATCC 13032. It is advantageous if the phenazine biosynthesis PhzD protein is tagged at its C- or N-terminus. If so, then the phenazine biosynthesis PhzD protein is preferably tagged with a short tag sequence with from 5 to 15 units, selected from the group consisting of His, Myc and Strep tags. Within this selection of tag groups, the phenazine biosynthesis PhzD protein is preferably His-tagged (with from 5 to 15 His-units) at its N-terminal end.

In addition, it is extremely advantageous if, in this embodiment of the process according to the invention, the expression of the phenazine biosynthesis PhzD protein is controlled by a T7 polymerase promoter upstream of the His-phzD gene. This is most surprising, since such effect of the T7 promoter is not to be expected, especially in host strains like Escherichia coli, where T7 polymerase is absent. Most preferably, the phenazine biosynthesis PhzD protein is His₁₀-tagged.

[3R,4R]-4-Amino-3-hydroxycyclohexa-1,5-diene-1-carboxylic acid (3,4-CHA) can be recovered from the fermentation broth obtained in this second embodiment of the present invention by any methods known to the skilled man. Preferably, it is recovered from the said fermentation broth by crystallization or by a separation process selected from the group consisting of reactive extraction and chromatography, optionally followed by crystallization.

The processes of the first and second embodiment of this invention can be carried out in vivo in a suitable host organism. When the processes according to the first and second embodiment of this invention are carried out in vivo, that is in living cells, prokaryotic as well as, if the biosynthetic pathway to chorismate is present therein, eukaryotic cells may be used as host cells for the process of the invention. It is to be noticed, that the process according to the second embodiment of this invention also may be carried out (enzymatically) in vitro.

The host organism for the in vivo processes according to the invention can be any host suitable for fermentation processes. Most preferably, however, the process (of the first or second embodiment of this invention) is performed in a host organism selected from the group of genera consisting of Bacillus, Corynebacterium, Escherichia, and Pichia.

The present invention further relates to expression vectors for use in a process according to any of the method claims of the present invention, where the process is carried out in vivo. As meant herein, expression vectors may comprise all of the required genes together on a single vector, or comprise different genes at different vectors. The vectors, or their expression cassettes (operons), or the genes pertaining thereto, may be chromosomally integrated.

The present invention, moreover, relates to host cells from one of the host organisms selected from the group of genera consisting of Bacillus, Corynebacterium, Escherichia, and Pichia, comprising at least one of the following activities or combinations of activities at an increased level of activity as compared to the level of native activity of such enzyme in its native surroundings under standard conditions, namely of
a PabAB bipartite protein as used according to any of the claims 3 to 8; or
an 4-amino-4-deoxychorismate synthase (preferably a PabAB bipartite protein), and a 4-amino-4-deoxyprephenate dehydrogenase and an aminotransferase; or
an 4-amino-4-deoxychorismate synthase (preferably a PabAB bipartite protein), and an enzyme capable of converting isochorismate into [5S,6S]-5,6-dihydroxycyclohexa-1,3-diene-1-carboxylic acid (2,3-CHD); or
an 4-amino-4-deoxychorismate synthase (preferably a PabAB bipartite protein), and of an enzyme capable of converting isochorismate into [5S,6S]-5,6-dihydroxycyclohexa-1,3-diene-1-carboxylic acid (2,3-CHD) and of converting 4-amino-4-deoxyisochorismate into [5S,6S]-6-amino-5-hydroxycyclohexa-1,3-diene-1-carboxylic acid (2,3-CHA); or
a PabAB bipartite protein and a phenazine biosynthesis PhzD protein; or
a PabAB bipartite protein and a tagged phenazine biosynthesis PhzD protein.

It is noted that, in references dealing with biosynthesis of p-aminobenzoate in Escherichia coli, combined overexpression of pabA and pabB genes has been described. Reference can be made to Ye et al., Proc. Natl. Acad. Sci., Vol. 87, p. 9391-9395, 1990, and to earlier work of Nichols et al., J. Biol. Chem., Vol. 264, p. 8597-8601,1989. The latter two documents, however, do not show nor suggest overexpression of a PabAB protein, nor combination thereof with an aminotransferase. Accordingly, the specific host cells listed above are novel.

Of course, within the context of the present invention, the hosts used also may contain further modifications to improve the biosynthesis of the desired products. For instance, genes encoding for activities that may be competing with the chorismate activity may be deleted (e.g. deletion of the genes tyrA, encoding chorismate mutase/prephenate dehydrogenase, and/or pheA, encoding chorismate mutase/prephenate dehydratase, from the chromosome); or key enzymes of the common aromatic pathway may be overexpressed (e.g. overexpression of aroF, encoding DAHP synthase).

The present inventors have further found, that the [3R,4R]-4-amino-3-hydroxycyclohexa-1,5-diene-1-carboxylic acid (3,4-CHA) prepared according to the second embodiment of the method of the present invention can very suitably be used as a catalyst, and in particular as a chiral catalyst, for instance in the aldol condensation of 4-nitrobenzaldehyde with acetone in the presence of a Zn-complex with 3,4-CHA. 3,4-CHA is a chiral molecule. The enantiomeric excess of the chiral product obtained in chiral catalysis with 3,4-CHA, for instance the e.e. of the product [S]-1-p-nitrophenyl-2-hydroxybutane-3-one, is surprisingly much higher than in the case of the known chiral catalysis reaction using the Zn-complex with L-proline as ligand. Various general reviews and articles teach asymmetric catalysis with compounds such as L-proline. For instance, see List, B., et al., J. Am. Chem. Soc., Vol. 122, p. 2395-2396, 2000; Dalko, P. I. et al., Angew Chem. Int. Ed. 40, p. 33726-22748, 2001; List, B., Tetrahedron 58, p. 5573-5590, 2002; Chandrasekhar, S., et al., Tetrahedron Lett., Vol. 45, p. 4581-4582, 2004; Berkessel, A., et al., Asymmetric Organocatalysis, VCH, Weinheim, 2004; Seayad, J., et al., Org. Biomol. Chem., Vol. 3, p. 719-724, 2005.

On the other hand it can be noted, that chiral catalysis using α-amino acids—also other amino acids than L-proline are shown—is known, for instance, from two recent publications, namely of: Darbre, T., et al., Chem. Commun., 2003, p. 1090-1091, showing catalysis by means of Zn-salts; and Amedjkouh, M., Tetrahedron: Asymmetry, Vol. 16, p. 1411-1414, 2005, showing use in, for instance, asymmetric aldol reactions.

None of the abovementioned reviews and articles, however, provides any teaching regarding the use of such amino acids and/or derivatives thereof for chiral catalysis, where the amino group is not located at an α- or β-position relative to the carboxylic group. It may, however, be noted, that β-amino acids (e.g. β-alanine) indeed already have been described (e.g. by Prout, F. S., in J. Org. Chem., Vol. 18, p. 928-933, 1953) as catalysts in the Knoevenagel reaction, a reaction that cannot be catalyzed by α-amino acids (such as α-alanine). But such disclosure cannot make the use of 3,4-CHA as a catalyst, and in particular as a chiral catalyst, obvious.

Accordingly and surprisingly, the present invention, in this further independent embodiment of the invention, also relates to the use of [3R,4R]-4-amino-3-hydroxycyclohexa-1,5-diene-1-carboxylic acid (3,4-CHA), obtained by the process according to the second embodiment of the invention, as a catalytically active product, in particular as a chiral catalyst.

Finally, the present invention also relates to the further conversion of [3R,4R]-4-amino-3-hydroxycyclohexa-1,5-diene-1-carboxylic acid (3,4-CHA) as can be produced according to the present invention. Such further conversion advantageously can be carried out to obtain all kinds of derivatives of 3,4-CHA. A good example of such further conversion is the synthesis of oseltamivir (phosphate).

Thus far, all syntheses of oseltamivir (phosphate) require the introduction of two nitrogen functionalities in position 4 and 5 of the target molecule with the correct relative and absolute configuration. The shortest route published so far, which uses shikimate as a starting compound, requires 11 steps. This is, schematically, represented in Scheme 1 below.

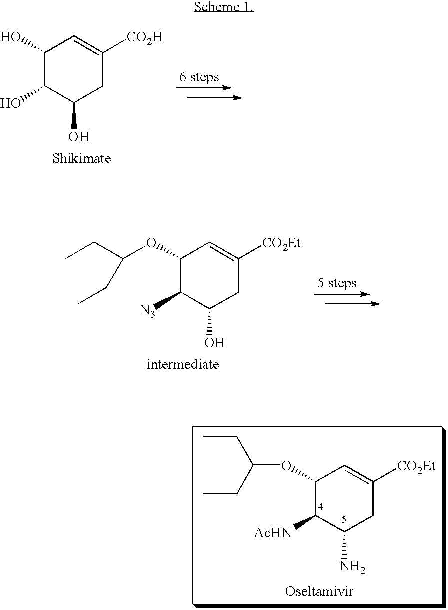

As can been seen from Scheme 2 below, ADC as well as 3,4-CHA contain the nitrogen functionality at C-4 in the right absolute and relative configuration with regard to the oxygen-substituent at C-3. Thus, the synthesis of oseltamivir (phosphate) according to the present invention, namely starting from the biosynthesis of 3,4-CHA, is highly simplified in that only the amino group at C-5 has to be introduced correctly. Introduction of the C-5 amino group can be performed e.g. via aziridination and subsequent reduction (cf. X. E. Hu, Tetrahedron, 60, p. 2701-2743 (2004)) or via amination, either directly or via, for instance, solvomercuration and subsequent reduction according to standard techniques (e.g. as described by M. B. Gasc et al., Tetrahedron 39, p. 703-731 (1983)). Protection of 3,4-CHA can be performed via standard techniques. Accordingly, after such shortened series of reaction steps, which may be carried out as a sequence of individual reaction steps that can be carried out in varying orders of steps, preferably (but not necessarily) via the fully protected compound that still has to be aminated at the C-5 position (referred to in Scheme 2 as “new intermediate”) as shown in Scheme 2 below. However, many different sequences of such individual reaction steps are possible starting from the 3,4-CHA prepared according to the present invention. Moreover, also 3,4-CHA prepared in another way than by biosynthesis according to the present invention can be used as starting material for the subsequent reaction steps leading to oseltamivir phosphate.

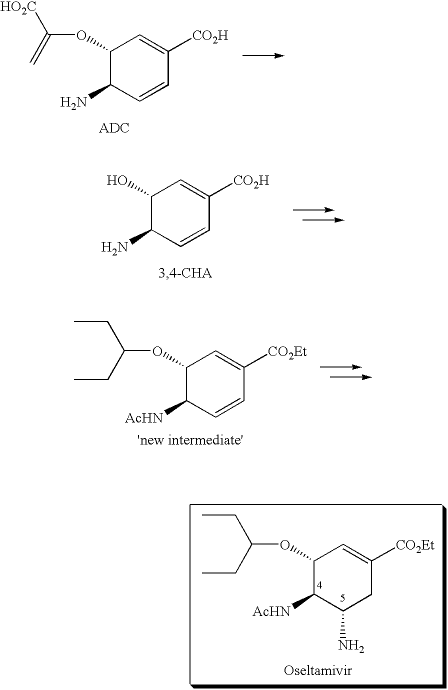

In particular, therefore, the present invention also relates to a novel method for the synthesis of oseltamivir phosphate, comprising the steps of
a) providing [3R,4R]-4-amino-3-hydroxycyclohexa-1,5-diene-1-carboxylic acid (3,4-CHA), preferably by biosynthesis of 3,4-CHA according to any of the claims relating to the synthesis of 3,4-CHA; and,
   in any order of steps b)-e)
b) esterification of the carboxylic function to its ethyl ester;
c) etherification of the hydroxylic function into its 3-pentanol ether;
d) acetylation of the amino group at C-3;
e) introduction of an amino function in trans-position with respect to the N-containing group at C-4; followed by
f) conversion of the product prepared from 3,4-CHA as a result of the combined reaction steps b), c), d), and e) into oseltamivir phosphate.

A reaction that, for instance, suitably can be applied in the step b), esterification, has been described by Federspiel et al, Organic Process & Research Development 1999, p. 266-274, especially the conversion of intermediate 23 into 24 in the first step of the shikimic acid route at page 273. Etherification in step c) may be done by all methods known to the skilled man. Acetylatation in step d), for instance, can be performed using a method described by Karpf et al., J. Org. Chem. 66, p. 2044-2051 (2001), in particular in the conversion of intermediate 17 into 18 at page 2049. Introduction of an amino function as in step e), for instance, may be carried out by aziridination or amination (see the article of X. E. Hu, respectively that of M. B. Gasc et al., mentioned above). The final conversion into oseltamivir phosphate, for instance, can be done as described by Carr et al., J. Org. Chem., 62, p. 8640-8653 (1997), in particular the conversion of intermediate 12 into 13 at page 8648.

The attached formulae sheet, preceding the experimental part, shows the structures of various of the compounds mentioned in this application, namely:

Chorismate

Isochorismate

3,4-CHA (trans position of amino and hydroxy group)

2,3-CHA (trans position of amino and hydroxy group)

3,4-CHD (trans position of hydroxy groups)

2,3-CHD (trans position of hydroxy groups)

ADC

ADIC

ADP

p-aminophenylalanine.

The invention hereinafter will be explained by means of a number of examples, which by no means are intended to restrict the scope of the present invention.

Sequence listings of nucleotides referred to in the experimental part, namely [SEQ ID: No. 1] to [SEQ ID: No. 10], are shown at the end of the experimental part. They also will be submitted in electronic format (PatentIn).

### Formulae Sheet

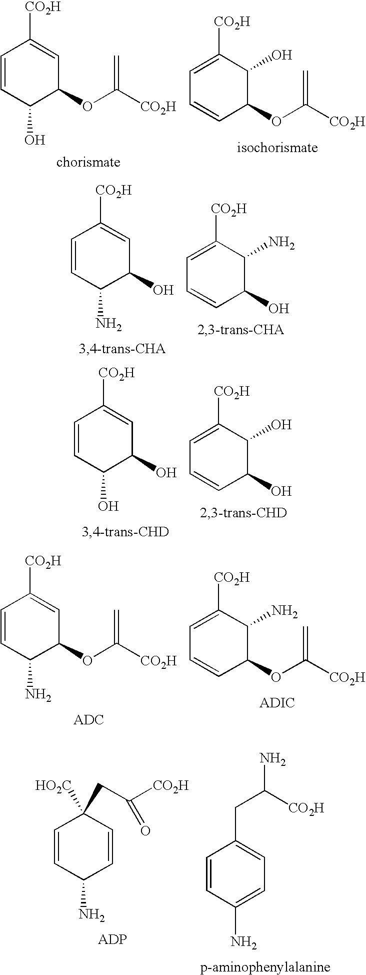

### EXPERIMENTAL PART

### Experimental Part I

### Examples 1-8

### Cloning of Single Genes and Construction of ADC and 3,4-CHA Biosynthesis Operons

### General Procedures for Examples 1-8

Standard molecular cloning techniques such as plasmid DNA isolation, gel electrophoresis, enzymatic restriction modification of nucleic acids, E. coli transformation etc. were performed as described by Sambrook et al., 1989, “Molecular Cloning: a laboratory manual”, Cold spring Harbor Laboratories, Cold Spring Harbor, N.Y. Synthetic oligodeoxynucleotides were obtained from MWG-Biotech AG (Ebersberg, Germany). DNA sequence analyses were performed by GATC Biotech AG (Konstanz, Germany) and AGOWA GmbH (Berlin, Germany).

### General Considerations for Examples 1-8

As expression vector for the ADC and 3,4-CHA biosynthesis genes, plasmid pJF119EH was chosen; Fürste, et al. (1986, Gene, 48:119-131). The expression system uses the IPTG inducible tac promoter and carries the lac repressor (lacl^{q} gene), which keeps the expression of the cloned foreign gene in the absence of the inducer extremely low. Additionally, the phzD gene of P. aeruginosa was cloned in expression vector pET16b (Calbiochem-Novabiochem GmbH, Schwalbach/Ts., Germany) producing a PhzD protein that contains an N-terminal His•Tag® sequence and the protease Factor Xa recognition site.

### Example 1

### Construction of Plasmid pC26

Genomic DNA from Pseudomonas aeruginosa ATCC 17933 was obtained from the American Type Culture Collection (ATCC), Manassas, Va., USA. A 706 bp fragment comprising the open reading frame (ORF) for PA1902, encoding the phenazine biosynthesis protein PhzD was amplified by PCR from the chromosomal DNA from Pseudomonas aeruginosa ATCC 17933 (nucleotides 3857-4480 of accession number AE004616; amplified region nucleotides 3840-4480) using the following primers:

[SEQ ID: No. 1]

5′- GAGCGCAAAGCTTCCCCTGGAGACCGTGGCATGAGC -3′

(with HindIII recognition site underlined) and

[SEQ ID: No. 2]

5′- GTGGGCAAAGCTTTCATTCCAGCACCTCGTCGGTGG -3′

(with HindIII recognition site underlined).

A list of all nucleotide sequences used in the context of the present application is presented in the SEQUENCE LISTING annexed hereto.

Correct size of the amplified fragment was confirmed by agarose gel electrophoresis and the fragment was purified from the gel. The fragment was digested with the enzyme HindIII to generate sticky ends. The plasmid pJF119EH was digested with HindIII and dephosphorylated. The two fragments were subsequently ligated and used for the transformation of chemically competent cells of E. coli DH5α (Invitrogen GmbH, Karlsruhe, Germany). The transformants were selected on LB agar plates containing 100 mg·l⁻¹ ampicillin. A plasmid showing the correct insert sequence (as confirmed by sequencing) was called pC26.

It is to be noticed that the phzD gene occurs twice in the genome of Pseudomonas aeruginosa. An exact copy can be found under accession number AE004838 nucleotides 2623-3246 (PA4213).

### Example 2

### Construction of Plasmid pC49

The P. aeruginosa phzD gene encoding the phenazine biosynthesis protein PhzD was subcloned in pET16b using PCR. The PhzD ORF was amplified using

[SEQ ID: No. 3]

5′- CCCCTGGAGACCGTCATATGAGCGGCATTCCCG -3′

(with NdeI recognition site underlined) and

[SEQ ID: No. 4]

5′- CCGGTTCTGCGTTCTGATTTGGATCCTATCAGGC -3′

(with BamHI recognition site underlined)
as primers and pC26 (see example 1) plasmid DNA as template.

Correct size (712 bp) of the amplified fragment was confirmed by agarose gel electrophoresis. The fragment and the plasmid pET16b were digested with NdeI and BamHI. The two fragments were ligated and transformed in E. coli DH5α. Transformants were selected on LB agar plates containing 100 mg·l⁻¹ ampicillin. A plasmid showing the correct insert sequence (as confirmed by sequencing) was called pC49.

### Example 3

### Construction of Plasmid pC53

Genomic DNA was prepared from Corynebacterium glutamicum ATCC 13032, which was obtained from the American Type Culture Collection (ATTC), Manassas, Va., USA. A 1907 bp fragment comprising the ORF (Cgl0997) for the aminodeoxychorismate synthase was amplified by PCR from Corynebacterium glutamicum ATCC 13032 chromosomal DNA (nucleotides 1052000-1053883 of accession number NC 003450; amplified region nucleotides 1052000-1053888) using the following primers:

[SEQ ID: No. 5]

5′- TTTTAGATCTGTGGTTTTGTCAGAGGATGTC -3′

(with BglII recognition site underlined) and

[SEQ ID: No. 6]

5′- TTGGATCCGTACGTCATGGaAATTCAAC -3′

(with BamHI recognition site underlined and changed nucleotide, indicated by lower case letter, in order to destroy the internal EcoRI recognition site).

Correct size of the amplified fragment was confirmed by agarose gel electrophoresis and the fragment was purified from the gel. The fragment was digested with the enzyme BglII and BamHI to generate sticky ends. The plasmid pJF119EH was digested with BamHI and dephosphorylated. The two fragments were subsequently ligated and used for the transformation of chemically competent cells of E. coli DH5α. The transformants were selected on LB agar plates containing 100 mg·l⁻¹ ampicillin. A plasmid showing the correct insert sequence (as confirmed by sequencing) was called pC53.

### Example 4

### Construction of Plasmid pC56

In this example, the cloned pabAB and phzD from examples 1 and 3 were combined in the expression vector pJF119EH. The pabAB gene in pC53 was excised from the expression vector by digestion with EcoRI and BamHI, and this DNA fragment containing the pabAB gene was purified by gel electrophoresis. Plasmid pC26 was digested with EcoRI and BamHI, ligated together with the PabAB EcoRI/BamHI fragment and transformed in E. coli DH5α. Transformants were selected on LB agar plates containing 100 mg·l⁻¹ ampicillin. A plasmid comprising the two genes in the correct order in pJF119EH (as confirmed by restriction mapping) was designated pC56.

### Example 5

### Construction of Plasmid pC78

In this example, the cloned pabAB and phzD from examples 2 and 3 were combined in the expression vector pJF119EH. The phzD gene in pC49 was excised from the expression vector by digestion with BglII and BamHI, and this DNA fragment containing the phzD gene was purified by gel electrophoresis. Plasmid pC53 was digested with BamHI, dephosphorylated, ligated together with the PhzD BglII/BamHI fragment and transformed in E. coli DH5α. Transformants were selected on LB agar plates containing 100 mg·l⁻¹ ampicillin. A plasmid comprising the two genes in the correct order in pJF119EH (as confirmed by restriction mapping) was designated pC78.

### Example 6

### Construction of Plasmid pF34

Genomic DNA was prepared from E. coli W3110 strain LJ110 (T. Zeppenfeld et al., J. Bacteriol. 182 (2000), pages 4443-4452). A 1213 bp fragment comprising the gene aroF for the DAHP synthase (tyr) was amplified by PCR from E. coli LJ110 chromosomal DNA (nucleotides 5872-6942 of accession number AE000346; amplified region nucleotides 5786-6965) using the following primers:

[SEQ ID: No. 7]

5′- CTATCGAATTCGAGCATAAACAGGATCGCC -3′

(with EcoRI recognition site underlined) and

[SEQ ID: No. 8]

5′- CACTTCAGCAACCAGTTCCCGGGGCTTCGC -3′

(with SmaI recognition site underlined).

Correct size of the amplified fragment was confirmed by agarose gel electrophoresis. The fragment and the plasmid pJF119EH were digested with EcoRI and SmaI. The two fragments were ligated and transformed in E. coli DH5α. Transformants were selected on LB agar plates containing 100 mg·l⁻¹ ampicillin. A plasmid showing the correct insert sequence (as confirmed by sequencing) was called pF34.

### Example 7

### Construction of Plasmids pC99, pC100, pC101

In this example, the cloned pabAB and phzD from examples 3, 4, and 5 were combined with aroF in the expression vector pJF119EH. The plasmid pF34 was digested with the enzymes SmaI and ScaI and the 5595 bp fragment was purified from the gel.

7.1 Construction of Plasmid pC99

The plasmid pC53 was digested with EcoRI and the sticky ends were filled in using Klenow enzyme. Subsequently, the pabAB gene was excised by digestion with ScaI and the 2774 bp DNA fragment was purified by gel electrophoresis. The 5595 bp DNA fragment from pF34 and the 2774 bp DNA fragment were ligated and transformed in E. coli DH5α. Transformants were selected on LB agar plates containing 100 mg·l⁻¹ ampicillin. A plasmid comprising the aroF and pabAB genes (as confirmed by restriction mapping) was designated pC99.

7.2 Construction of Plasmid pC100

The plasmid pC56 was digested with EcoRI and the sticky ends were filled in using Klenow enzyme. Subsequently, the pabAB-phzD DNA fragment was excised by digestion with ScaI and the 3421 bp DNA fragment was purified by gel electrophoresis. The 5595 bp DNA fragment from pF34 and the 3421 bp DNA fragment were ligated and transformed in E. coli DH5α. Transformants were selected on LB agar plates containing 100 mg·l⁻¹ ampicillin. A plasmid comprising the aroF, pabAB, and phzD genes (as confirmed by restriction mapping) was designated pC100.

7.3 Construction of Plasmid pC101

The plasmid pC78 was digested with EcoRI and the sticky ends were filled in using Klenow enzyme. Subsequently, the pabAB-phzD DNA fragment was excised by digestion with ScaI and the 3613 bp DNA fragment was purified by gel electrophoresis. The 5595 bp DNA fragment from pF34 and the 3613 bp DNA fragment were ligated and transformed in E. coli DH5α. Transformants were selected on LB agar plates containing 100 mg·l⁻¹ ampicillin. A plasmid comprising the aroF, pabAB, and phzD genes (as confirmed by restriction mapping) was designated pC101.

### Example 8

### Construction of Host Strain F4

The pheA tyrA aroF gene locus of E. coli W3110 strain LJ110 (T. Zeppenfeld et al., J. Bacteriol. 182 (2000), pages 4443-4452) was inactivated according to K. A. Datsenko and B. L. Wanner, PNAS 97 (2000), pages 6640-6645. The inactivation cassette was amplified by PCR from the plasmid pKD4 using the following primers:

[SEQ ID: No. 9]

5′- AACTTCGTCGAAGAAGTTGAAGAAGAGTAGTCCTTTATATTGAGTG
TATCGT-GTAGGCTGGAGCTGCTTC- 3′

[SEQ ID: No. 10]

5′- AGTGTAAATTTATCTATACAGAGGTAAGGGTTGAAAGCGCGACTAA
ATTGCA-TATGAATATCCTCCTTAG- 3′

A strain carrying the deletion of the pheA tyrA aroF gene locus was designated F4.

### Experimental Part II

### Examples 9-12

### Production of ADC+ADP, and 3,4-CHA

### Example 9

### Fermentative Production of Mixture of ADC and ADP in Fed-Batch Bioreactor

The ADC production of E. coli F4/pC99 from glucose was investigated in mineral medium. The precultivation medium consisted of MgSO₄.7H₂O (0.3 g·l⁻¹), CaCl₂.2H₂O (0.015 g·l⁻¹), KH₂PO₄ (3.0 g·l⁻¹), K₂HPO₄ (12.0 g·l⁻¹), NaCl (0.1 g·l⁻¹), (NH₄)₂SO₄ (5.0 g·l⁻¹), FeSO₄.7H₂O (0.075 g·l⁻¹), Na-citrate.3H₂O (1.0 g·l⁻¹), thiamine.HCl (0.0125 g·l⁻¹), L-tyrosine (0.05 g·l⁻¹), and L-phenylalanine (0.05 g·l⁻¹). Additional minerals were added in the form of a trace element solution (1.0 ml·l⁻¹), which trace element solution was composed of Al₂(SO₄)₃.18H₂O (2.0 g·l⁻¹), CoCl₂.6H₂O (0.75 g·l⁻¹), CuSO₄.5H₂O (2.5 g·l⁻¹), H₃BO₃ (0.5 g·l⁻¹), MnCl₂.4H₂O (20.0 g·l⁻¹) Na₂MoO₄.2H₂O (3.0 g·⁻¹), NiSO₄.6H₂O (20 g·l⁻¹), ZnSO₄.7H₂O (15.0 g·l⁻¹). A glucose stock solution (500 g·l⁻¹) was autoclaved separately and added to the sterilized medium to a final concentration of 15 g·l⁻¹.

The stock culture of E. coli F4/pC99 was stored at −80° C. in Luria-Bertani (LB) medium containing 50% glycerol. 1 ml feedstock was used to inoculate 200 ml of precultivation medium containing ampicillin (100 mg·l⁻¹) split in two 1 l shaking flasks and incubated at 37° C. and 180 rpm for 16 hours.

The fermentation medium was the same as for precultivation except for the following changes: MgSO₄.7H₂O (0.9 g·l⁻¹), K₂HPO₄ was omitted, FeSO₄.7H₂O (0.1125 g·l⁻¹), Na-citrate.3H₂O (1.5 g·l⁻¹), thiamine.HCl (0.075 g·l⁻¹), L-tyrosine (0.15 g·l⁻¹), and L-phenylalanine (0.3 g·l⁻¹). Additional minerals were added in the form of a trace element solution (1.5 ml·l⁻¹).

Fed-batch cultivation was performed in 5 l Labfors bioreactor (Infors, Einsbach, Switzerland) with 10% inoculation for 27.25 h; 2 l initial volume, cultivation temperature 37° C., pH 6.8. pH was controlled by 12.5% ammonia and 2.5N KOH titration. After 8.25 h a feed for L-tyrosine and L-phenylalanine (5 g·l⁻¹ L-tyrosine, 6 g·l⁻¹ L-phenylalanine dissolved in 1N KOH; feed rate 7 g·h⁻¹) was started. After 9.25 h a feed for glucose (500 g·l⁻¹; feed rate 12.5 g·h⁻¹) was started. After 7.5 h at an OD_{620 nm} of ˜4.0 ADC production was induced by addition of 0.1 mM IPTG.

A sample of the culture supernatant was lyophilized and re-dissolved in D₂O. 600 MHz ¹H-NMR at 303 K showed the expected resonance spectrum. The assignment of all the resonances was done by using several 2D NMR techniques (¹H-¹H COSY, ¹H-¹H TOCSY, ¹H—¹³C COSY and ¹H—¹³C long range HMBC). All the spectra confirmed the presence of ADC and ADP. The amount of ADC present was determined to be 7 g·l⁻¹. (Besides ADC and ADP, 16 g·l⁻¹ of its precursor chorismate was present as determined by HPLC analysis.)

In separate batches mixtures of ADC and ADP were isolated from fermentation supernatant by ion exchange chromatography (Dowex 50Wx8, H⁺-form, elution with 0.5 M ammonia solution) in varying ratios of ADC and ADP, often with an excess of ADP. Chorismate was removed quantitatively. The final product contained roughly 1:1 of ADC and ADP.

### Example 10

### In Vitro Production of 3,4-CHA

Single colonies of E. coli strain BL21 (DE3)pLysS (Calbiochem-Novabiochem GmbH, Schwalbach/Ts., Germany) harboring the plasmid pC49 (see Example 2) were cultivated in 200 ml LB medium containing ampicillin (100 mg·l⁻¹) at 37° C. At OD_{600 nm} of 0.6, the cells were induced by the addition of IPTG (1 mM). After 4 h the cells were harvested, resuspended in buffer (20 mM N-(2-hydroxyethyl)-piperazine-N′-(2-ethanesulfonic acid (HEPES), 300 mM NaCl, 1 mM dithiothreitol (DTT), 20 mM imidazole) and frozen at −70° C. for later use.

The frozen cells were thawed at 30° C. in a water bath and then incubated for 1 h on ice for complete lysis. Cell debris was removed by centrifugation and the resulting cell free extract was applied onto a 25 ml Ni-NTA Superflow column (Qiagen, Hilden, Germany). After a washing step (20 mM HEPES, 300 mM NaCl, 1 mM DTT, 20 mM imidazole) the His•PhzD protein was obtained in at least 95% purity by elution with buffer (20 mM HEPES, 300 mM NaCl, 1 mM DTT) containing increasing concentrations of imidazole (max. 250 mM). The partially purified extract was used in further investigation.

The 3,4-CHA production assay of 3 ml contained 0.2 M K-phosphate buffer (pH 7.6), 85 mg ADC, and 62.5 μg partially purified His•PhzD. The assay was started by addition of His•PhzD and stopped after 2 h at 37° C. The protein was removed by centrifugation through a Centricon 10 column (Milipore, Eschborn, Germany). The reaction mixture was separated by ion exchange chromatography. A sample was dissolved in D₂O and analyzed by 300 MHz ¹H-NMR at 293K confirming the presence of 3,4-CHA.

### Example 11

### Fermentative Production of 3,4-CHA in Shake Flask

The 3,4-CHA production of E. coli strains F4/pC100 and F4/pC101, respectively, from glucose was investigated in mineral medium. This mineral medium was the same as the precultivation medium as described in example 9, except for glucose (10 g·l⁻¹).

The stock cultures of E. coli F4/pC100 and F4/pC101 were stored at −80° C. in Luria-Bertani (LB) medium containing 50% glycerol. 1.8 ml feedstock was used to inoculate 50 ml of minimal medium containing ampicillin (100 mg·l⁻¹) in a 500 ml shaking flask and incubated at 37° C. and 180 rpm for 16 hours. 125 μl of this culture was subsequently used to inoculate 50 ml of the same medium in a 500 ml shaking flask and incubated at 37° C. and 180 rpm for 24 h. After 3.25 h at an OD_{620 nm} of ˜1.5, the cells were induced by adding 0.1 mM IPTG.

A sample of the culture supernatant was lyophilized and re-dissolved in D₂O. 600 MHz ¹H-NMR at 303 K showed the expected resonance spectrum. The assignment of all the resonances was done by using several 2D NMR techniques (¹H-¹H COSY, ¹H-¹H TOCSY, ¹H—¹³C COSY and ¹H—¹³C long range HMBC). All the spectra confirmed the presence of 3,4-CHA. The amount present was determined to be 30 mg·l⁻¹ and 150 mg·l⁻¹ for the strains F4/pC100 and F4/pC101, respectively. (Besides 3,4-CHA, 102 mg·l⁻¹ and 520 mg·l⁻¹, respectively, of 3,4-CHD were present, but no ADC, ADP, and p-aminobenzoic acid as determined by HPLC and NMR analysis.)

### Example 12

### Fermentative Production of 3,4-CHA in Fed-Batch Bioreactor

The precultivation of F4/pC101 was performed as described in example 9. The fermentation medium was the same as in example 9.

Fed-batch cultivation was performed in 5 l Labfors bioreactor (Infors, Einsbach, Switzerland) with 10% inoculation for 31.25 h; 2 l initial volume, cultivation temperature 37° C., pH 6.8. pH was controlled by 12.5% ammonia and 2.5N KOH titration. After 7.5 h a feed for L-tyrosine and L-phenylalanine (5 g·l⁻¹ L-tyrosine, 6 g·l⁻¹ L-phenylalanine dissolved in 1N KOH; feed rate 7 g h¹) was started. After 6.5 h a feed for glucose (500 g·l⁻¹; feed rate 12.5 g·h⁻¹) was started. After 7.0 h at an OD_{620 nm} of ˜7.5, 3,4-CHA production was induced by addition of 0.1 mM IPTG. The supernatant was analyzed for 3,4-CHA by ¹H-NMR and the amount present was determined to be 1.7 g·l⁻¹. Besides said amount of 3,4-CHA, 5.4 g·l⁻¹ of 3,4-CHD was found to be present, as well as very minor amounts, at about their detection limits, of ADC (<37 mg·l⁻¹) and of p-aminobenzoic acid (<27 mg·l⁻¹) were present as determined by HPLC analysis.

3,4-CHA was isolated, in a yield of about 90% by weight, from fermentation supernatant by ion exchange chromatography.

All sequences of nucleotides referred to in the experimental part are listed hereinafter, and will be submitted in electronic format (PatentIn).

### Experimental Part III

### Examples 13 and 14; Comparative Examples A and B

### Use of 3,4-CHA, Obtained According to the Invention, as a Catalyst

### Example 13

### Catalysis with Pure 3,4-CHA Obtained According to the Said Embodiment of the Invention (and Comparative Example A: Same Reaction with L-proline)

The inventors have demonstrated by means of a model reaction (Knoevenagel condensation analogous to the ones described in Cardillo, G., et al., Synth. Commun., Vol. 33, p. 1587-1594), and in comparison to the same reaction using L-proline as a catalyst, that 3,4-CHA itself can be used as a catalyst for said reaction.

### Comparative Example A

3-Methylbutyraldehyde (1.55 mmol; 172 μl of a liquid having a density of 0.801 g/ml) was dissolved in DMSO, and L-proline (0.2 mmol; 23.0 mg) was added. Then, after 5 minutes, dimethyl malonate (4 mmol; 459 μl of a liquid having a density of 1.156 g/ml) was added and the mixture was stirred over night at room temperature (r.t.). The reaction mixture was diluted with ethyl acetate and washed twice with water. The organic layer was dried over anhydrous Na₂SO₄. 297 mg of the condensation product, dimethyl 2-(3-methyl-butylidene)malonate, was obtained. Accordingly, the yield of the condensation product was 96%.

### Example 13

3-Methylbutyraldehyde (1.55 mmol; 172 μl of a liquid having a density of 0.801 g/ml) was dissolved in DMSO, and 3,4-CHA (0.2 mmol; 31.0 mg) was added. Then, after 5 minutes, dimethyl malonate (4 mmol; 459 μl of a liquid having a density of 1.156 g/ml) was added and the mixture was stirred for 70 hours at room temperature (r.t.). The reaction mixture was diluted with ethyl acetate and washed twice with water. The organic layer was dried over anhydrous Na₂SO₄. 183 mg of the condensation product, dimethyl 2-(3-methyl-butylidene)malonate, was obtained. Accordingly, the yield of the condensation product was 46%.

### Example 14

### Chiral Catalysis with Zn-Complex of Pure 3,4-CHA Obtained According to the Said Embodiment of the Invention (and Comparative Example B: Same Reaction with L-Proline)

The inventors have demonstrated by means of a model reaction (aldol condensation using zinc-complexes of 3,4-CHA analogous to the syntheses described by Darbre, T., et al. Chem. Commun., p. 1090-1091, 2003), and in comparison to the same reaction using the zinc-complexes of L-proline as a catalyst, that zinc-complexes of 3,4-CHA show asymmetric catalytical activity, for instance in the formation of 4-hydroxy-4-(4-nitrophenyl)butan-2-one. Good e.e.'s can be obtained for the said reaction product.

No conversion at all was observed when the same reaction was performed using only zinc acetate as a catalyst.

### Comparative Example B

L-Proline (4.34 mmol; 499 mg) was dissolved in MeOH followed by addition of triethylamine (TEA; 0.6 ml of a liquid having a density of 0.73 g/ml). The mixture was stirred for 10 min at room temperature (rt). Then, zinc acetate (2.17 mmol; 476 mg) was added to the mixture, and a colourless precipitate appeared immediately after the said addition. After stirring for 1 h the solid material was filtered and dried to get 532 mg (41%) of the zinc-(L-proline)₂ complex. This complex was characterized by the following ¹H-NMR (D₂O) characteristics: δ=1.87 (br s, 3H), 2.29 (br m, 1H), 3.04 (br s, 1H), 3.19 (br m, 1H), 3.92 (br s, 1H).

A small amount of the above zinc-(L-proline)₂ complex in water (0.05 mmol; 15 mg of the complex in 10 ml of water) was then added to a solution of 4-nitrobenzaldehyde (1.0 mmol; 151 mg) in 5 ml of acetone, and the reaction mixture was stirred at rt under inert conditions (N₂). After 4 h, the reaction mixture was evaporated and the residue was dissolved in chloroform. All non-soluble material was filtered. The filtrate was then rotatory evaporated to give 173 mg (83% yield, 11% e.e.) of 4-hydroxy-4-(4-nitrophenyl)butan-2-one. Determination of the e.e. was done by chiral phase HPLC (Chiralpak AS, Daicel).

This product was characterized by the following ¹H-NMR (D₂O) characteristics: δ=2.23 (s, 3H), 2.86 (m, 2H), 3.61 (br s, 1H, OH), 5.28 (dd, J=7.7, 4.6 Hz, 1H), 7.55 (d, J=9.1 Hz, 2H), 8.22 (d, J=9.1 Hz, 2H); respectively by the following ¹³C-NMR (D₂O) characteristics: δ=30.9 (CH₃), 51.7 (CH₂), 69.1 (CH), 124.0 (2×CH), 126.6 (2×CH), 150.1 (2×C_{q}), 208.8 (C═O).

### Example 14

3,4-CHA (1.08 mmol; 167 mg) was dissolved in MeOH, and the same amount of water was added, followed by addition of triethylamine (TEA; 1.08 mmol; 150 μl of a liquid having a density of 0.73 g/ml). The mixture was stirred for 10 min at rt. Then, zinc acetate (0.54 mmol; 119 mg) was added to the mixture. After stirring for 1 h the solid material was filtered. From the filtrate, after addition of 240 μl of TEA, a white solid precipitated, which was separated and dried to give 104 mg (30% yield) of a zinc-(3,4-CHA)₂ complex.

A small amount of the above zinc-(3,4-CHA)₂ complex in water (0.05 mmol; 18.8 mg of the complex in 10 ml of water) was then added to a solution of 4-nitrobenzaldehyde (1.0 mmol; 151 mg) in 5 ml of acetone, and the reaction mixture was stirred at rt under inert conditions (N₂). After 70 h, the reaction mixture was evaporated and the residue was dissolved in chloroform. The non-soluble material was filtered. The filtrate was then rotatory evaporated to give 90 mg (43% product, 88% e.e.) of 4-hydroxy-4-(4-nitro-phenyl)butan-2-one. Determination of the e.e. was done by chiral phase HPLC (Chiralpak AS, Daicel).

It is to be noted, that the e.e. of this product showed almost no dependency of reaction time and conversion: after 42 h of reaction time and at about 24% conversion, the product even was enantioenriched at a level of 90% e.e.

1. Process for the biosynthetic production of 4-amino-4-deoxychorismate (ADC) catalyzed at least by an enzyme belonging to the class of aminodeoxychorismate synthases, characterized in that the biosynthetic production is performed fermentatively in vivo in a host microorganism with a 4-amino-4-deoxychorismate synthase at an increased level of activity, while obtaining a fermentation broth comprising 4-amino-4-deoxychorismate (ADC) and 4-amino-4-deoxyprephenate (ADP), and that these compounds, either together or individually, are recovered from the fermentation broth.

2. Process according to claim 1, characterized in that the 4-amino-4-deoxychorismate synthase is a PabAB bipartite protein.

3. Process according to claim 2, characterized in that the bipartite protein originates from a species from the group of Actinomycetes, or from plants containing such bipartite enzyme, or is constructed by fusion of genes respectively encoding for PabA and PabB.

4. Process according to claim 2, characterized in that the PabAB bipartite protein originates, as such or as a fusion protein, from a species from the group of genera consisting of Escherichia, Corynebacterium, Saccharomyces and Streptomyces.

5. Process according to claim 4, characterized in that the PabAB bipartite protein originates, as such or as a fusion protein, from one of the species from the group of species consisting of Escherichia coli, Corynebacterium glutamicum, Corynebacterium diphtheriae gravis NCTC13129, Corynebacterium efficiens, Saccharomyces cerevisiae, Streptomyces griseus, Streptomyces venezuelae, Streptomyces sp. FR-008, Streptomyces pristinaespiralis, Streptomyces thioluteus, and Streptomyces avermitilis.

6. Process according to claim 5, characterized in that the PabAB bipartite protein originates from Corynebacterium glutamicum ATCC 13032.

7. Process according to claim 1, characterized in that the 4-amino-4-deoxychorismate (ADC) and 4-amino-4-deoxyprephenate (ADP) formed are recovered from the said fermentation broth, together or individually, by a separation process selected from the group consisting of reactive extraction and chromatography, optionally followed by crystallization.

8. Process for the biosynthetic production of p-aminophenylalanine integrated in a biosynthetic process for the production of 4-amino-4-deoxychorismate (ADC) catalyzed at least by an enzyme belonging to the class of aminodeoxychorismate synthases, characterized in that the biosynthetic production is performed fermentatively in vivo in a host microorganism with a 4-amino-4-deoxychorismate synthase at an increased level of activity, while obtaining a fermentation broth comprising 4-amino-4-deoxychorismate (ADC) and 4-amino-4-deoxyprephenate (ADP), and in that the 4-amino-4-deoxyprephenate (ADP) in the mixture of 4-amino-4-deoxychorismate (ADC) and 4-amino-4-deoxyprephenate (ADP) is converted into p-aminophenylalanine by means of concerted action, and at an increased level of activity, of a 4-amino-4-deoxyprephenate dehydrogenase and an aminotransferase, and in that p-aminophenylalanine is recovered from the fermentation broth.

9. Process for the biosynthetic production of [3R,4R]-4-amino-3-hydroxycyclohexa-1,5-diene-1-carboxylic acid (3,4-CHA) catalyzed at least by an enzyme belonging to the class of aminodeoxychorismate synthases, characterized in that the biosynthetic production is performed by concerted action, and at an increased level of activity, of a 4-amino-4-deoxychorismate synthase and of an enzyme capable of converting isochorismate into [5S,6S]-5,6-dihydroxycyclohexa-1,3-diene-1-carboxylic acid (2,3-CHD), and that the [3R,4R]-4-amino-3-hydroxycyclohexa-1,5-diene-1-carboxylic acid (3,4-CHA) is recovered from the fermentation broth so obtained.

10. Process according to claim 9, characterized in that the biosynthetic production is performed fermentatively in vivo in a host microorganism.

11. Process according to claim 9, characterized in that the 4-amino-4-deoxychorismate synthase is a PabAB bipartite protein.

12. Process according to claim 11, characterized in that the PabAB bipartite protein is a protein obtained by a biosynthetic production performed fermentatively in vivo in a host microorganism with a 4-amino-4-deoxychorismate synthase at an increased level of activity, while obtaining a fermentation broth comprising 4-amino-4-deoxychorismate (ADC) and 4-amino-4-deoxyprephenate (ADP), and that these compounds, either together or individually, are recovered from the fermentation broth, and in that the enzyme capable of converting isochorismate into [5S,6S]-5,6-dihydroxycyclohexa-1,3-diene-1-carboxylic acid (2,3-CHD) is also capable of converting 4-amino-4-deoxyisochorismate into [5S,6S]-6-amino-5-hydroxycyclohexa-1,3-diene-1-carboxylic acid (2,3-CHA).

13. Process according to claim 11, characterized in that the enzyme capable of converting isochorismate into [5S,6S]-5,6-dihydroxycyclohexa-1,3-diene-1-carboxylic acid (2,3-CHD) is a phenazine biosynthesis PhzD protein.

14. Process according to claim 13, characterized in that the phenazine biosynthesis PhzD protein originates from a species from the group of genera consisting of Pseudomonas, Pantoea, Streptomyces, and Erwinia.

15. Process according to claim 13, characterized in that the phenazine biosynthesis PhzD protein originates from a species selected from the group consisting of Pseudomonas aeruginosa, Pseudomonas aureofaciens, Pseudomonas fluorescens, Pseudomonas chlororaphis, and Pantoea agglomerans species.

16. Process according to claim 13, characterized in that the phenazine biosynthesis PhzD protein originates from Pseudomonas aeruginosa ATCC 17933.

17. Process according to claim 11, characterized in that the PabAB bipartite protein originates from a species selected from the group of species consisting of Escherichia coli, Corynebacterium glutamicum, Corynebacterium diphtheriae gravis NCTC13129, Corynebacterium efficiens, Saccharomyces cerevisiae, Streptomyces griseus, Streptomyces venezuelae, Streptomyces sp. FR-008, Streptomyces pristinaespiralis, Streptomyces thioluteus, and Streptomyces avermitilis.

18. Process according to claim 17, characterized in that the PabAB bipartite protein originates from Corynebacterium glutamicum ATCC 13032.

19. Process according to claim 9, characterized in that the [3R,4R]-4-amino-3-hydroxycyclohexa-1,5-diene-1-carboxylic acid (3,4-CHA) formed is recovered from the said reaction mixture by crystallization or by a separation process selected from the group consisting of reactive extraction and chromatography, optionally followed by crystallization.

20. Process according to claim 1, characterized in that the process is performed in a host organism selected from the group of genera consisting of Bacillus, Corynebacterium, Escherichia, and Pichia.

21. Expression vector for use in a process according to claim 1.

22. Host cells from one of the host organisms selected from the group of genera consisting of Bacillus, Corynebacterium, Escherichia, and Pichia, comprising at least one of the following activities or combinations of activities at an increased level of activity as compared to the level of native activity of such enzyme in its native surroundings under standard conditions, namely of
a PabAB bipartite protein as used according to claim 3; or
an 4-amino-4-deoxychorismate synthase (preferably a PabAB bipartite protein), and a 4-amino-4-deoxyprephenate dehydrogenase and an aminotransferase; or
an 4-amino-4-deoxychorismate synthase (preferably a PabAB bipartite protein), and
an enzyme capable of converting isochorismate into [5S,6S]-5,6-dihydroxycyclohexa-1,3-diene-1-carboxylic acid (2,3-CHD); or
an 4-amino-4-deoxychorismate synthase (preferably a PabAB bipartite protein), and of an enzyme capable of converting isochorismate into [5S,6S]-5,6-dihydroxycyclohexa-1,3-diene-1-carboxylic acid (2,3-CHD) and of converting 4-amino-4-deoxyisochorismate into [5S,6S]-6-amino-5-hydroxycyclohexa-1,3-diene-1-carboxylic acid (2,3-CHA), or
a PabAB bipartite protein and a phenazine biosynthesis PhzD protein; or
a PabAB bipartite protein and a tagged phenazine biosynthesis PhzD protein.

23. Use of [3R,4R]-4-amino-3-hydroxycyclohexa-1,5-diene-1-carboxylic acid (3,4-CHA), obtained by the process according to claim 9, as a catalytically active product, in particular as a chiral catalyst.

24. Method for the synthesis of oseltamivir phosphate, comprising the steps of
a) providing [3R,4R]-4-amino-3-hydroxycyclohexa-1,5-diene-1-carboxylic acid (3,4-CHA),
and, in any order of steps b)-e)
b) esterification of the carboxylic function to its ethyl ester;
c) etherification of the hydroxylic function into its 3-pentanol ether;
d) acetylation of the amino group at C-3;
e) introduction of an amino function in trans-position with respect to the N-containing group at C-4;
followed by
f) conversion of the product prepared from 3,4-CHA as a result of the combined reaction steps b), c), d), and e) into oseltamivir phosphate.


SEQUENCE LISTING (10 sequences)

SEQ ID NO: 1 - DNA, 36 bp, Artificial Sequence
  primer
gagcgcaaagcttcccctggagaccgtggcatgagc

SEQ ID NO: 2 - DNA, 36 bp, Artificial Sequence
  primer
gtgggcaaagctttcattccagcacctcgtcggtgg

SEQ ID NO: 3 - DNA, 33 bp, Artificial Sequence
  primer
cccctggagaccgtcatatgagcggcattcccg

SEQ ID NO: 4 - DNA, 34 bp, Artificial Sequence
  primer
ccggttctgcgttctgatttggatcctatcaggc

SEQ ID NO: 5 - DNA, 31 bp, Artificial Sequence
  primer
ttttagatctgtggttttgtcagaggatgtc

SEQ ID NO: 6 - DNA, 28 bp, Artificial Sequence
  primer
ttggatccgtacgtcatggaaattcaac

SEQ ID NO: 7 - DNA, 30 bp, Artificial Sequence
  primer
ctatcgaattcgagcataaacaggatcgcc

SEQ ID NO: 8 - DNA, 30 bp, Artificial Sequence
  primer
cacttcagcaaccagttcccggggcttcgc

SEQ ID NO: 9 - DNA, 70 bp, Artificial Sequence
  primer
aacttcgtcgaagaagttgaagaagagtagtcctttatattgagtgtatcgtgtaggctggagctgcttc

SEQ ID NO: 10 - DNA, 70 bp, Artificial Sequence
  primer
agtgtaaatttatctatacagaggtaagggttgaaagcgcgactaaattgcatatgaatatcctccttag